(19) United States
(12) Patent Application Publication
(10) Pub. No.: US 2009/0101845 A1
(43) Pub. Date: Apr. 23, 2009

(54) Exposure Device for Printing Plates

(75) Inventors: Sascha Neber, Adendorf (DE); John Hedde, Luneburg (DE)

Correspondence Address: COLLARD & ROE, P.C., 1077 NORTHERN BOULEVARD, ROSLYN, NY, 11576, US

(73) Assignee: Punch Graphix Prepress Germany GmbH

(21) Appl. No.: 11/887,598

(22) PCT Filed: Mar. 31, 2006
(86) PCT No.: PCT/EP2006/002962

§ 371 Date: Oct. 1, 2007

(30) Foreign Application Priority Data
Apr. 2, 2005 (DE) 10 2005 015 192.2
Apr. 2, 2005 (DE) 10 2005 015 193.0

(51) Int. Cl. A61N 5/067

(52) U.S. Cl. 250/492.1

(57) ABSTRACT

The invention relates to an exposure device (1) which comprises a light source (2), a light modulator (4) in addition to a light coupling unit (3). The aim of the invention is to optimize, in a simple and economical manner, the radiation intensity in the spectral area which is necessary for exposing the material (6) which is to be exposed. The light source (2) comprises at least one laser diode (7).

The present invention relates to an exposure device having a light source, a light modulator, as well as a light coupling unit.

An exposure device of the type stated is described, for example, in DE 698 22 004/EP 1 141 780. This known exposure device comprises a lamp with a capacitor arrangement, a light modulator composed of a reflected micromirror arrangement, as well as suitable optics to guide the light emitted by the lamp over the light modulator onto the medium to be exposed. An arc lamp or incandescent lamp having an oval beam spot is used as the lamp. Divergent radiation that is processed further by capacitor optics proceeds from the oval beam spot.

It is a disadvantage of the previously known exposure device that the gas discharge lamp or incandescent lamp selected as a light source emits light over a broad frequency spectrum. In contrast, in the case of UV exposure devices, for example, only a small frequency range in the ultraviolet to violet range, in other words approximately 350 nm to 450 nm, can be used for the actual exposure. For this reason, a large part of the radiation emitted by the lamp must be considered lost power for the exposure system. This consequently results in a whole number of problems, such as the need for cooling to remove the heat power, for example, and an unnecessarily high power consumption, in comparison with the useful power, and the high operating costs connected with this.

Another disadvantage of the previously known exposure device is furthermore the aforementioned divergence of the radiation emitted by the light source. Because of the divergence of the emitted light, the circumstance occurs that the light can only be used in part for the actual exposure process, in the spectral range that is fundamentally usable, in other words, in the case of UV exposure, of 350 nm to 450 nm. This is attributable to the fact that the light source has a greater etendue than the other components of the exposure device, because of the relatively large oval light spot and the aforementioned divergence. Therefore not all of the light of the light source can be coupled into the system. In total, the power loss of the system is therefore increased once again. Furthermore, there are frequently problems caused by the scattered light that is not coupled into the exposure system.

A device for exposing printing plates is also known from DE 195 45 821 A1, having an exposure head, a light source, an image production unit, as well as imaging optics. In the case of this exposure device, the use of a metal halogen lamp is also provided. This lamp has the disadvantages described above.

To circumvent the problems stated, it would also be possible, according to the state of the art, to implement the exposure device with a single laser, e.g. a gas laser or solid-body laser. It is true that the emission could be restricted to a narrow usable frequency band in this way, on the one hand, and on the other hand, a very slight divergence of the emitted light could be achieved.

However, a disadvantage of the use of a single laser is that the latter is generally very cost-intensive in terms of its acquisition. Furthermore, there is the problem, when using a single laser, that the coherence length of the emitted light is very long, and this can lead to undesirable refraction phenomena in the exposure device, particularly at the location of the medium to be exposed. The refraction, in turn, can lead to undesirable non-homogeneity in the illumination of the light modulator, which is a disadvantage, whereas a uniformly homogeneous illumination of the light modulator is aimed at. When a single laser is used for the exposure device, it therefore has to be additionally assured, in the design, that no interferences occur. This generally has a negative effect on costs.

The present invention is therefore based on the task of indicating an exposure device of the type stated initially, in which the irradiation intensity is optimized in the spectral range that is required for exposure of the material to be exposed, in particularly simple and cost-advantageous manner.

This task is accomplished, according to the invention, in that the light source comprises a laser diode, in the case of an exposure device of the type indicated. Laser diodes have been available in cost-advantageous manner for quite some time, with radiation emission characteristics over a broad wavelength range. Laser diodes having radiation emission characteristics in specifically the wavelength range of 350 nm to 450 nm are easily available on the market, particularly for the structured exposure of UV-sensitive material, or material sensitive to violet light, in other words in the range of 350 nm to 450 nm.

According to the invention, the laser diodes can be single-mode or multi-mode. The light of a laser diode is restricted to a desired, narrow frequency interval, in desired manner, on the one hand; on the other hand, it is advantageously assured, by means of the integrated resonator, that the emitted light is highly bundled, in other words demonstrates little divergence; finally, however, in this connection the coherence length of the light emitted by the laser diode according to the invention is significantly less than is the case for lasers, such as gas lasers or solid-body lasers, for example.

The low coherence length of the laser diodes according to the invention has the great advantage that no problems with undesirable refraction effects occur within the exposure device according to the invention, which effects would in turn lead to a non-homogeneous illumination of the light modulator.

According to an advantageous embodiment of the exposure device according to the invention, the laser diode emits light in the range of approximately 350 nm to approximately 450 nm. In this way, the exposure of conventional and violet offset printing plates, in particular, is made possible in advantageous manner. In the same way, the exposure device according to the invention can advantageously be used for exposing screens for screen printing, flexo printing plates, proof materials, as well as steel plates for punch pattern production, in structured manner. Furthermore, it is advantageous that for some time, laser diodes have been available in this wavelength range at relatively advantageous prices.

In a further advantageous embodiment of the exposure device according to the invention, the light source comprises a module of several, preferably twenty laser diodes. In this way, the illumination intensity available for exposure in the relevant wavelength range can be further increased, in advantageous manner, in that the light of the module comprising several laser diodes is added. This is fundamentally possible, since the etendue of each individual laser diode is significantly less than the etendue of the exposure system, which results from the display and the projection optics. Therefore the total light of the module can have an etendue, by means of suitable addition of the individual laser diodes of the module according to the invention, that is still less than the etendue of the exposure system resulting from the display and the projection optics. In the end result, the light of all the laser diodes belonging to the module can therefore fundamentally be coupled completely into the exposure device, in advantageous manner. In this connection, there are no fundamental physical restrictions caused by the etendue.

Another advantage of the use of a module consisting of several laser diodes is a greater measure of availability of the system, since the failure of a single laser diode allows continuation of the exposure process with the exposure device according to the invention, if the system is suitably designed.

The use of twenty laser diodes makes it possible to use components available on the market, and has proven to be particularly advantageous in the design of the exposure device according to the invention.

If, according to another advantageous embodiment of the exposure device according to the invention, a beam flow of each laser diode in the light coupling unit is geometrically superimposed, the light of several individual laser diodes can be used for illuminating one and the same light modulator unit. The radiation intensity of the material to be exposed is further optimized in this manner.

In an advantageous embodiment of the invention, it is provided that the light coupling unit comprises individual glass fibers. Coupling the light emitted by the laser diode into individual glass fibers is not a problem, since the radiation of the laser diode is emitted from a very small exit facet. Furthermore, transport of the light coupled in, to the light modulator and other components of the exposure device according to the invention, is advantageously possible without problems once the light has been coupled in. Furthermore, further homogenization of the light coupled in takes place in the interior of the individual glass fibers, by means of multiple reflection at the border surfaces of the fibers, between the fiber entrance and fiber exit.

In an embodiment of the invention, it is provided that each laser diode has a separate individual glass fiber assigned to it. When using several laser diodes, for example a module of laser diodes, the individual fibers can easily be brought together, so that a geometric addition of the radiation emitted by the individual laser diodes is achieved. The light of each individual laser diode is furthermore homogeneously mixed with the light of the other laser diodes, by means of multiple reflection at the border surfaces of the fibers, between the fiber entrance and the fiber exit. In this way, an extremely homogeneous illumination of the light modulator of the exposure device according to the invention is advantageously achieved.

If, in a special embodiment of the invention, the individual glass fiber has a diameter of approximately 125 μm, one obtains a particularly advantageous embodiment, which allows almost loss-free coupling in of the light of the individual laser diode, whereby the dimensions of the light coupling unit can nevertheless be kept small, in advantageous manner.

If, in a further development of the invention, the individual glass fibers are brought together into a fiber bundle in the light coupling unit, geometrical superimposition of the light emission of each individual laser diode is also advantageously possible, so that the light that exits at the end of the fiber bundle acts like a single light source, with the added intensity of each individual laser diode.

It has proven to be particularly advantageous, according to the invention, if the outside diameter of the fiber bundle amounts to approximately 650 μm.

To further increase the radiation intensity of the light-sensitive material, the light source can comprise several modules, according to the invention. In this connection, each module in turn consists of several, for example of twenty laser diodes. Again, in this connection, advantageous use is made of the circumstance that the etendue of each individual laser diode is significantly less than the etendue of the system of display and projection optics. On the other hand, when using conventional light sources such as gas discharge lamps, for example, the situation is precisely the opposite, i.e. the etendue of the gas discharge lamp is significantly greater than the etendue of the system of display and projection optics. In the case of an exposure device such as a gas discharge lamp, the use of several light sources to increase the radiation intensity would fundamentally be possible only with restrictions. However, in the case of the present invention, this problem is circumvented by means of the use of laser diodes, with great advantage.

According to an alternative advantageous embodiment variant of the exposure device according to the invention, the light coupling unit comprises a light-integrating glass rod. Light-integrating glass rods have the advantage that if their cross-section is suitably selected, e.g. if a rectangular cross-section is selected, very good homogenization of the light coupled into the glass rod is possible. For example, it would be possible to couple the light of several individual laser diodes according to the invention into a glass rod having a rectangular cross-section and a suitable average area.

Very good mixing of the light of the individual laser diodes then advantageously takes place by means of the reflection processes at the inner border surface of the glass rod to the surroundings, so that an illumination of the light modulator unit with homogeneous light of high spectral intensity is possible at the exit from the glass rod.

Furthermore, the possibility of geometrically adding the light of several individual light sources, according to the invention, is based on the property of the individual laser diodes to demonstrate an extremely low etendue. At the same time, refraction effects that disrupt homogeneity are generally also not to be expected in the case of superimposition of the individual laser diodes, because of the relatively short coherence length.

In a specific further development of the invention, the glass rod has an angular, preferably rectangular cross-section. The advantage is that the rectangular rod cross-section can be geometrically adapted, at the exit, to a rectangular light modulator, for example a DMD™ (Digital Micromirror Device).

According to a particularly preferred further development of the exposure device according to the invention, the radiation flow is configured to be variable. In this way, the result is advantageously achieved that the exposure device can be adapted, by the user, to different materials or applications. For example, certain applications can require a lower exposure intensity than others. Very flexible adaptation of the process parameters is achieved by means of the variable configuration of the radiation flow of the light source, in interplay with the exposure time.

In this connection, it has proven to be advantageous if the radiation flow of the laser diode itself is configured to be variable.

In this way, direct variation of the radiation flow of the light source of the exposure device according to the invention can be achieved. In this connection, the variation can be implemented essentially in stepless manner, by means of the use of power sources that can be suitably regulated.

In an alternative embodiment of the variant of the invention, the light source is provided with means for separately switching individual laser diodes on and off. When using a module of several laser diodes for the light source, a variation of the radiation flow can advantageously be achieved in this way, while keeping the operating current constant. In this connection, the radiation flow can fundamentally be varied in steps from 100% to 0%, whereby the number of gray levels that can be achieved is determined by the number of individual laser diodes within a module. In many application cases, such a stepped variability is sufficient, and it is advantageously possible to do without power regulation to vary the radiation flow.

The use of the light source according to the invention is particularly advantageous in the case of an exposure device having a light source, a light modulator having a plurality of lines of light-modulating cells, a device for imaging data patterns on the light modulator, a device for imaging the light modulator on light-sensitive material, a device for producing a relative movement between the light modulator and the light-sensitive material, whereby the relative movement runs essentially perpendicular to the lines of light-modulating cells, as well as having a device for scrolling a data pattern displayed on a given line of the light modulator through the various lines of the light modulator, whereby the scrolling takes place synchronized with the relative movement, in such a manner that imaging of a data pattern displayed on the light modulator, on the light-sensitive material, is essentially stationary with reference to the light-sensitive material, since the exposure times can be reduced due to the higher light intensity.

Such an exposure device is known, for example, from EP 0 095 4624.3 (corresponds to PCT/EP 00/07842). There, a system is disclosed, which is particularly used in processes in which large amounts of modulated light in the blue and ultraviolet range are needed, such as in the exposure of printing plates, and the exposure of printed circuit boards, for example.

The principle of the exposure devices that function according to the so-called Integrating Digital Screen Imaging method (IDSI), is that the light-sensitive material is continuously moved, while the image content is scrolled through the light modulator at the same speed, in the opposite direction. The image content therefore remains fixed in place on the material to be exposed. In this connection, the exposure time of each pixel results from integration of all of the relatively short individual exposures of the same cell of a row. The exposure time depends on the process speed, the duty cycle of the light modulator, and the number of lines. At a given process speed and a given duty cycle, the exposure time furthermore depends on the number of lines. The greater the number of lines of the light modulator, the longer the exposure times that can be achieved, if each data pattern is moved (scrolled) from the line at an outer side of the light modulator over the entire display region of the light modulator all the way to the outer line that lies opposite to the starting line.

For process optimization reasons, the greatest possible minimization of the exposure times is aimed at. For this purpose, the light sensitivity of the light-sensitive material can be increased, for example. In this case, it would fundamentally be possible to increase the relative movement between the light modulator and the light-sensitive material in the case of the previously known exposure device. The exposure time for each pixel, integrated up over all the lines of the light modulator, is then shortened accordingly. In this connection, of course, the scrolling rate must be increased synchronously with the relative movement.

Available light modulators are microdisplay devices that are composed, in each instance, of a matrix of individually controllable individual light modulators. In this connection, digital mirror arrangements (DMD™ “Digital Micromirror Device”), liquid crystal matrices (LCDs) having a transmissive architecture, or also, for some time now, reflective liquid crystal displays (LCOS), are particularly widespread.

All of these available light modulators have in common that they modulate the image data in pixels. In particular, these light modulators do not contain any shift register that would make it possible for image data for a line that have been transmitted to the light modulator to be independently transmitted further (shifted) into the adjacent line, in each instance. Therefore, the complete display has to be completely written to again, in each instance, in order to implement the scrolling principle, if the exposure unit moves by one image line relative to the light-sensitive material. Therefore image data for every pixel of the light modulator matrix have to be transmitted to the scrolling procedure for every individual exposure state.

As a result, very high data transmission rates occur in practice. For example, the data to be transmitted can reach the order of 10 GBit/s. In contrast, however, the available light modulator matrices are limited, with regard to the data transmission rates to be received by them. For example, the data transmission rate of a light modulator matrix can be limited to 7.6 Gbit/s.

Because of this, a great disadvantage of the known exposure devices can occur, with regard to the minimal exposure times that can actually be achieved. If the scrolling rate that belongs to a maximally permissible data transmission rate to the light modulator matrix leads to exposure times defined by the number of lines, which exceeds the minimal exposure time required by the light-sensitive material, the minimal exposure time defined by the light-sensitive material actually cannot be achieved by the exposure device.

In this case, the minimal exposure time is therefore not determined by the light-sensitive material, but rather is established, in undesirable manner, by the exposure device and, in particular, by the limitation of the maximal data transmission rate to the light modulator matrix. The exposure time of an exposure device of the type stated is furthermore determined by the product of the number of lines of the light modulator and the reciprocal image rate of the scrolling process.

U.S. Pat. No. 5,672,464 also discloses an exposure device of the type stated, also based on the scrolling principle. There, the case is discussed that the available number of lines of the light modulator matrix exceeds the number of sequential exposures required for complete exposure of the light-sensitive material. For this case, it is proposed to extend the scrolling process not over all of the lines of the light modulator, in that individual lines are switched out or switched to black. However, this is supposed to take place by means of transmission of corresponding data patterns to the light modulator, whereby the image data for the “black” lines also have to be transmitted. Therefore there is no reduction in the amount of data to be transmitted.

Thus, the method and the exposure device from U.S. Pat. No. 5,672,464 also demonstrate the restriction of the minimal exposure time that can be technically implemented, as described above. Also, the minimal exposure time that can be technically implemented is not determined by the photochemical sensitivity of the light-sensitive material, for example, but instead is limited, in undesirable manner, by the maximally possible data transmission rate of image data to the light modulator.

The advantages of the light source according to the invention can be better utilized if a device for inactivating a number of lines of the light modulator, which number can be predetermined, is provided, and the light modulator can be operated by charging essentially only the active lines with the data patterns. In this way, the light modulator is advantageously limited to a reduced number of activated lines, while the pixels of the inactivated lines are permanently switched to the “OFF” switching status, without data having to be transmitted to the light modulator for this purpose.

Because the light modulator can additionally be operated in such a manner that it merely requires the image data, in other words the data patterns, for the actively set lines, but not the data for the inactivated lines, for these actively set lines, a higher scrolling rate can be set at a given data transmission rate to the light modulator.

This is advantageously achieved, not by means of a technically complicated increase in the data transmission rate to the light modulator, for example, but instead by means of a reduction of the image data to be transmitted. In this way, the maximal exposure speed can be brought close to the value that is predetermined by the minimally required exposure time, in accordance with the photochemical properties of the light-sensitive material to be exposed, by means of a suitable selection of the number of lines to be inactivated.

It is particularly advantageous, in a specific embodiment of the exposure device according to the invention, if the device is configured for inactivation of a cohesive region of lines, which region can be predetermined. As compared with a plan according to which one switches all of the odd or all of the even lines inactive, for example, optimization of the illumination of the active line region can advantageously be implemented in the case of the inactivation of cohesive regions of lines, according to the invention. Furthermore, it is generally advantageous for the exposure process if the exposure takes place cohesively, in one piece, and not with interruptions in accordance with lines inserted in between, which have been switched to be inactive.

A particularly flexible, advantageous embodiment of the exposure device according to the invention is obtained if the region of the lines switched inactive is configured to be shiftable. In this way, the useful lifetime of the light modulator matrix can be increased, among other things, in that the region of active lines is shifted to a line region of the light modulator in which no or as few as possible pixels have failed, induced by radiation. Since it is not necessary, according to the invention, to use all of the lines of the light modulator at the same time, it is therefore possible, according to the invention, to continue to use the light modulator without restrictions for the image quality, by means of shifting the lines to be switched inactive to such lines that contain failed pixels, induced by radiation.

In a further development of the exposure device according to the invention, means for focusing the light source essentially on the active lines of the light modulator are provided. In this way, a further reduction in the exposure time can be achieved, since the beam density of the light radiation that falls on the active lines is increased. While the portion of the light radiation that falls on the lines set to be inactive is lost, when some lines of the light modulator are set to be inactive, without the focusing of the light source on the active cells, according to the invention, according to this variant, which is in accordance with the invention, all of the light power of the light source is used exclusively for illuminating the actively set lines of the light modulator, which are actually in use.

In order to ensure that the illuminated region of the light modulator always coincides with the actively set line region of the light modulator, particularly also for the case that the region of the active lines of the light modulator varies, it is advantageous, according to a specific embodiment of the invention, if the means for focusing are configured to be variable with regard to a focal region.

According to a particularly advantageous variant of the invention, the light source has a smaller etendue than a unit formed from the light modulator and the device for imaging the light modulator on light-sensitive material. In this way, it is ensured that when the light source is focused on the region of the active lines of the light modulator, the radiation emitted by the light source is nevertheless completely available for exposure of the light-sensitive material, despite the increase in the divergence of the beams that unavoidably accompanies this, for reasons of imaging optics. In this case, there is no loss of light radiation that can be used for the exposure, due to limitations in the numerical aperture of the subsequent optical components of the exposure device.

The invention will be described as an example in a preferred embodiment, making reference to a drawing, whereby other advantageous details can be derived from the figures of the drawing.

In this connection, parts that are the same in terms of function are indicated with the same reference symbols.

The figures of the drawing show, in detail:

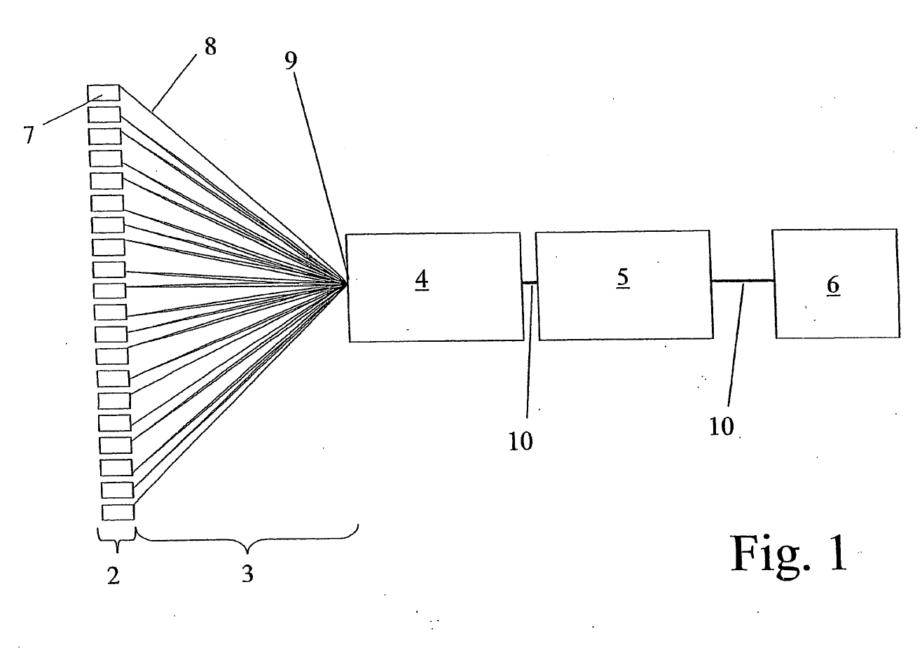

FIG. 1 schematic representation of the overall structure of the preferred embodiment of the exposure device according to the invention,

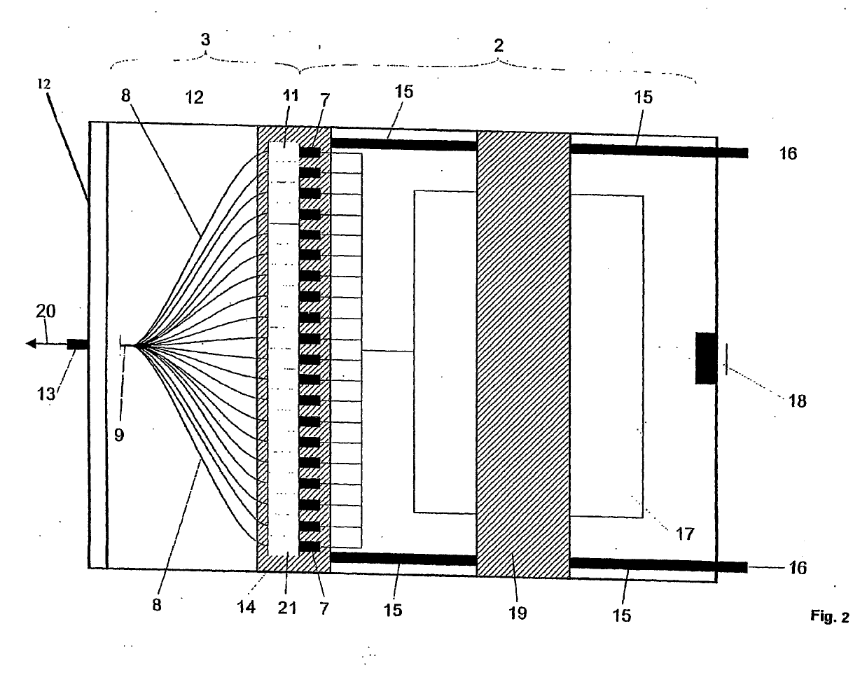

FIG. 2 schematic detail representation of the light source and the light coupling unit of the exposure device according to the invention from FIG. 1,

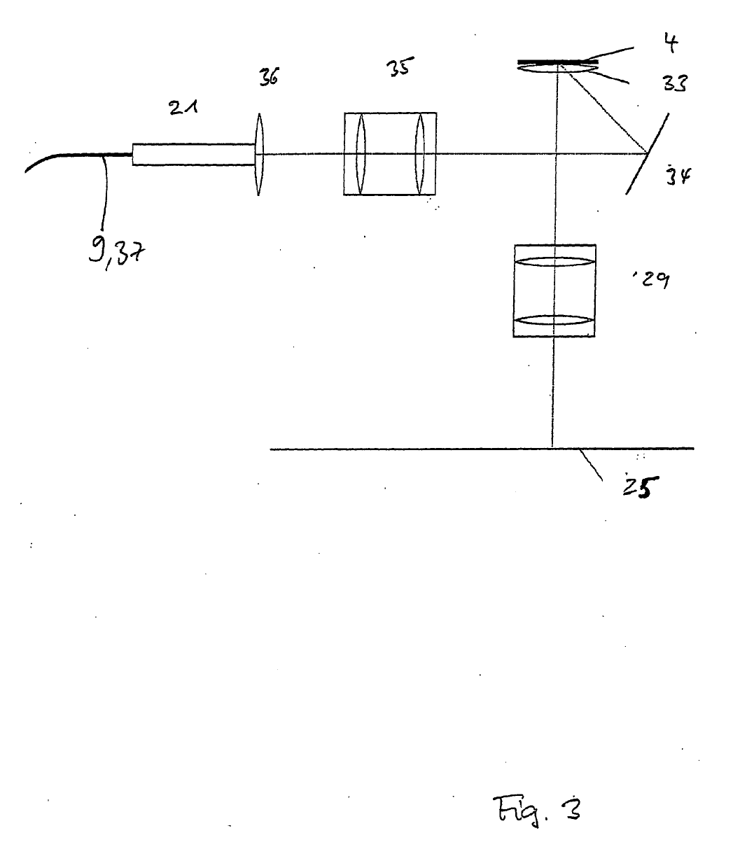

FIG. 3 schematic representation of the beam path in the exposure head,

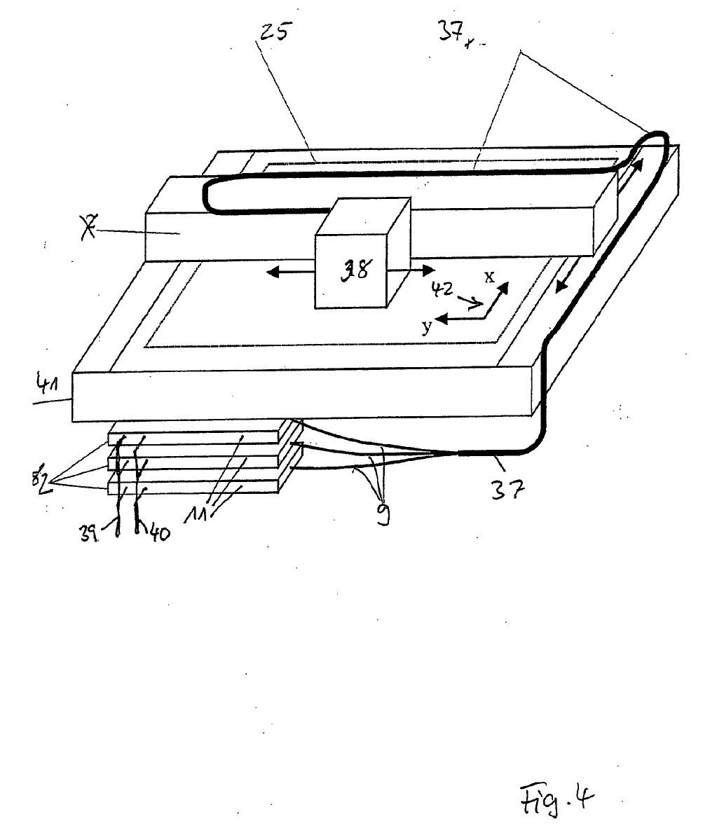

FIG. 4 schematic representation of the exposure device with a stationary light source,

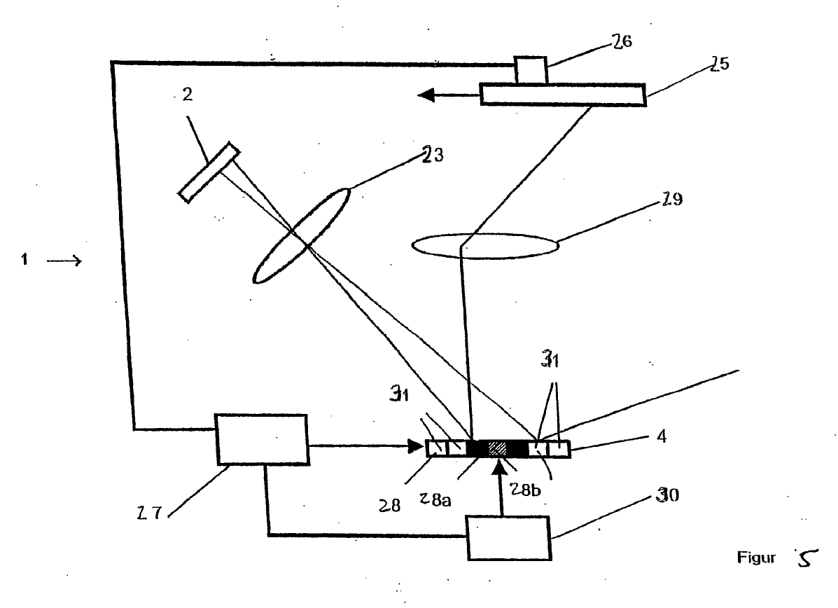

FIG. 5 a schematic representation of the complete exposure device according to the invention,

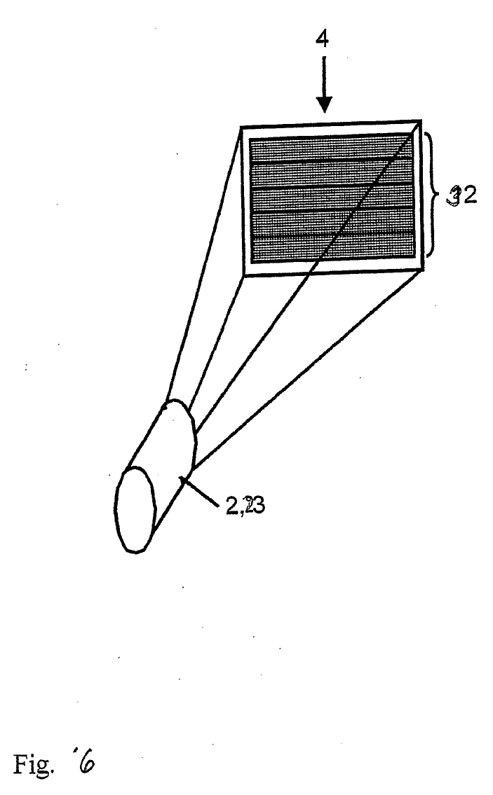

FIG. 6 a schematic representation of the fundamental method of operation of a conventional exposure device according to the state of the art,

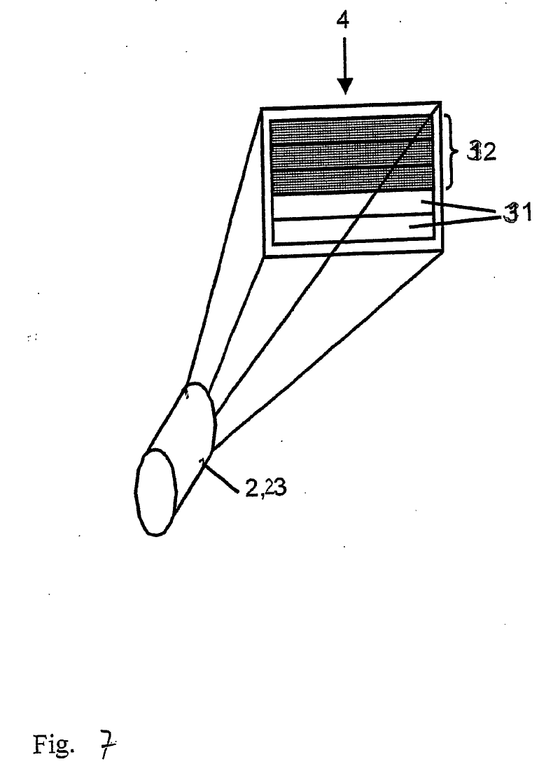

FIG. 7 a schematic representation of the principle of operation of the exposure device according to the invention,

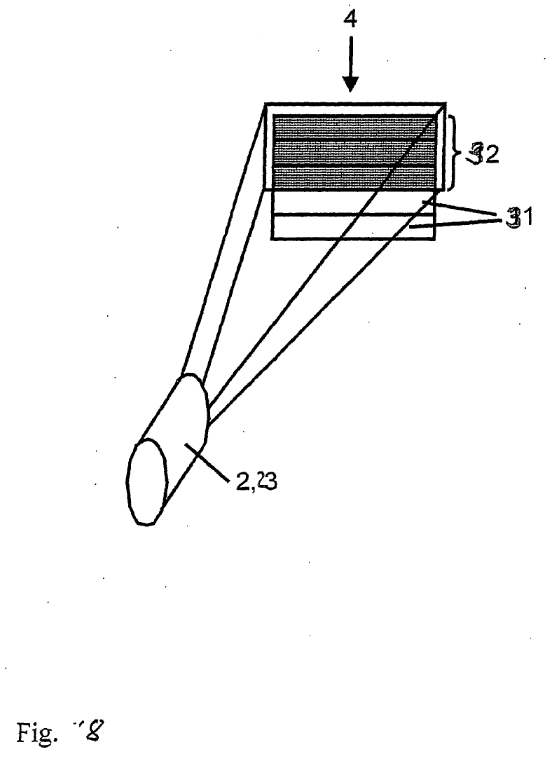

FIG. 8 a schematic representation of the principle of operation of another embodiment of the exposure device according to the invention.

FIG. 1 schematically shows an exposure device 1 according to the invention. The exposure device 1 consists of a light source 2, a light coupling unit 3, a light modulator 4, and imaging optics 5. Furthermore, a material 6 to be exposed can be seen in FIG. 1. The light source 2 is composed of a line of a total of twenty individual laser diodes 7. Each laser diode 7 emits light in the wavelength range of approximately 400 to 410 nm. The optical radiation power of each laser diode 7 amounts to approximately 60 mwatts. The light of each individual laser diode 7 is coupled into the light coupling unit 3. In this connection, the light coupling unit 3 consists of a total of twenty individual light fibers 8. Each individual light fiber 8 has exactly one laser diode 7 assigned to it, so that the light of each laser diode 7 is coupled into one individual glass fiber 8, in each instance. The individual glass fibers 8 run together and are brought together into a fiber bundle 9 at the entrance of the light modulator 4. The light modulator 4 is illuminated with the fiber bundle 9. In this connection, light from each of the twenty individual laser diodes 7 falls on the light modulator 4 at the exit from the fiber bundle 9. The diameter of each individual glass fiber 8 amounts to 125 μm. The total outside diameter of the fiber bundle 9 amounts to 650 μm. The light 10 modulated by the light modulator 4 is passed to the material 6 to be exposed, by way of the imaging optics 5.

The very small etendue of the individual laser diodes 7, in comparison with the etendue of the overall system of the light modulator 4 and the imaging optics 5, has the result that the light that exits at the exit of the fiber bundle 9 in turn has an etendue that is still less than the etendue of the system of light modulator 4 and imaging optics 5. Thus, in theory, loss-free coupling of the light of all twenty laser diodes into the illumination device 1 according to the invention is possible.

FIG. 2 shows some details of the unit of laser diodes 7 and light coupling unit 3, according to the invention. As can be seen, the light source 2 comprises a total of twenty laser diodes 7. The laser diodes 7 are brought together into a module 11 consisting of twenty individual laser diodes 7. At the exit of the module 11, each laser diode 7 has an individual glass fiber 8 assigned to it. The twenty individual glass fibers 8, in total, are brought together into the fiber bundle 9. The fiber bundle 9 is passed through the outer housing 12 of the unit of light source 2 and light coupling unit 3, and is connected with an FC (fiber channel) plug 13 there.

The module 11, which comprises the twenty laser diodes 7, is affixed to a cooling plate 14. The cooling plate 14 is connected with a cooling pipe 15 at the ends, in each instance. Cooling fluid can be filled into the cooling pipe 15 and circulated, at the connector piece 16.

The light source 2 furthermore comprises the control unit 17 for the twenty laser diodes 7. The control unit 17 of the light source 2 is supplied with energy by way of a plug 18 having the D-sub format. The control unit 7 is connected with the cooling body 19 in heat-conductive manner. The cooling body 19, in turn, is connected with the cooling pipes 15 in heat-conductive manner.

At the exit of the FC plug 13, the total emission of the twenty laser diodes 7 of the module 11 is emitted as total radiation 20. The total radiation 20 has an etendue that is less than the etendue of the unit of light modulator 4 and imaging optics 5. Therefore, there are no fundamental restrictions with regard to the possibility of coupling the total radiation 20 into the light modulator 4 and the imaging optics 5. Thus, the essential part of the total radiation 20 can be directed at the material 6 to be exposed.

In the operation of the light source 2, cooling fluid is passed through the cooling pipe 15 by way of the connector pieces 16. In this connection, the cooling fluid that circulates in the cooling pipe 15 transports heat away from the control unit 17 of the laser diode 7, by way of the cooling body 19. In addition, the cooling fluid in the cooling pipe 15 conducts heat away from the module 11, which consists of the twenty laser diodes 7, by way of the cooling plate 14. In total, the waste heat conducted away is significantly less, in relation to the usable emitted total radiation 20 according to the invention, than is the case for conventional exposure devices, which use a gas discharge lamp or incandescent lamp having a broad emission spectrum as the light source.

In this way, an exposure device is proposed, according to the invention, with which the radiation intensity of UV-sensitive material 6 is optimized in comparatively simple and cost-advantageous manner. In the design of the imaging optics 5, no refraction effects generally have to be taken into consideration, since the coherence length of the laser diodes 7 is comparatively short. Homogenization of the light emitted by the individual laser diodes takes place by means of multiple reflection within the individual glass fibers 8 of the fiber bundle 9.

In FIG. 3, the beam path provided in the exposure head 38 can be seen. The light fed to the movable exposure head by way of the fiber bundle 9 or the beam waveguide 37, respectively, is first coupled into the integrator rod 21. The integrator rod 21 serves for homogenization of the light and is therefore also referred to as an integrating mixing rod. So-called hollow conductors or light tunnels can also be used, in which the light experiences a plurality of reflections. The light then leaves the integrator rod 21 through the integrator lens 36 and the lens system 35, causing the beam to be widened up. The mirror 34 then deflects the beam in the direction of the light modulator. As a result, the light passes through the field lens 33 twice, and then hits the light-sensitive material 25 by means of the imaging optics 29. The imaging optics 29 form the light of the light modulator 4 in the plane of the light-sensitive material 25.

FIG. 4 shows the possible structure of the exposure device according to the invention. In this connection, the light source 2 consists of several modules 11, which are all cooled by way of the cooling water inflow 39 and the cooling water outflow 40. The fiber bundles 9 that exit from the modules 11 are coupled into a beam waveguide 37 that feeds the light to the exposure head 28. The exposure head 38 moves in two orthogonally disposed axes 42. The beam waveguide 37 must follow this movement accordingly. The light-sensitive material 25 lies on the machine bed 41. Because of the external arrangement of the light source, the mass of the exposure head 38 can be advantageously reduced, so that the device demonstrates particularly good dynamics. When using a fiber laser or a disk laser, it is even possible to do without water cooling, in certain cases. In such cases, it can be advantageous to dispose the light source 2 in the exposure head, as well.

An exposure device 1 is schematically shown in FIG. 5. A light source 2 is imaged using first exposure optics 23 and a light modulator 4. The position of the light-sensitive material 25 relative to the light modulator 4 is changed by means of a position transmitter 26. The relative movement takes place perpendicular to the lines of the light modulator 4. Data patterns are transmitted into the first column with cells 28 of the light modulator, using a driver circuit 27. The cells of the light modulator 4 run perpendicular to the plane of the drawing, in the schematic representation.

What is important in the transmission of the data pattern into the first column with cells 28 of the light modulator 4 is the synchronization of the data pattern transmission with the movement of the light-sensitive material 25. The data pattern transmitted into the first line is shifted into the next line synchronous to the relative movement, so that the data pattern to be transmitted onto the light-sensitive material 25 remains fixed in place on the latter.

The light modulator 4 consists of several lines of cells 28. These run perpendicular to the plane of the drawing in this schematic representation. Each cell 28 in the schematic representation therefore corresponds to a line in the top view.

The data pattern to be transmitted to the light modulator 4 consists of combinations of light modulator cells 28 that are turned on 28a and turned off 28b. If the cells 28 are turned on, the light that falls on them is guided to the light-sensitive material 25 by way of second imaging optics 29. The light that hits cells 28b that are turned off is deflected away from the light-sensitive material 25. Up to this point, the exposure device 1 as described corresponds to the state of the art.

Furthermore, however, the present exposure device 1 according to the invention has a device 30 for the inactivation of a certain number of lines of the light modulator, which number can be selected. In this way, the number of cells 28 that must be charged with image data, i.e. data pattern data, by the driver circuit 27, is limited to the number of remaining lines of the light modulator 4 that have been switched to be active.

In the schematic representation, the lines switched to be inactive are designated with the reference symbol 31 and shown not filled out. The turned-off light modulator cells 28b, which are shown cross-hatched in the schematic representation, and the turned-on light modulator cells 28a, which are shown filled out in the schematic representation, must be distinguished from the lines 31 that are set to be inactive.

During the scrolling process, the data pattern data, in other words whether the light modulator cell 28 is supposed to be turned on or turned off, is transmitted by way of the driver circuit 27, for the turned-on light modulator cells 28a and the turned-off light modulator cells 28b, in each instance. In contrast, no data pattern transmission by the driver circuit 27 has to take place for the lines 31 set to be inactive by means of the device 30 for inactivation of lines. The amount of data to be transmitted to the light modulator 4 by the driver circuit 27 is thereby reduced by the image data for the lines 31 set to be inactive, which no longer have to be transmitted, according to the invention. The scrolling speed that can be achieved increases accordingly.

In normal operation, as known from the state of the art, a data pattern is first set by the driver circuit 27, for example to the line containing the turned-on light modulator cell 28a, and subsequently, the data pattern of this line is shifted to the adjacent line. Shifting takes place in that a completely new image is transmitted to the light modulator 4, in which image the line is shifted accordingly. Because this shift of the data patterns from line to line takes place parallel and synchronous to the relative movement of the light-sensitive material 25, which is produced by the position transmitter 26, the data pattern remains fixed in place on the light-sensitive material 25 in this process. The exposure period of the light-sensitive material 25 results from the number of active light modulator cells 28a, 28b, and the scrolling cycle rate, in other words the dwell time of an individual data pattern in a line.

Furthermore, it can be seen in FIG. 5 that the light source 2, by way of the illumination optics 3, merely illuminates the light modulator cells 28a, 28b that are fundamentally active, i.e. for which data pattern data are transmitted to the light modulator 4 by way of the driver circuit 7. In contrast, the light source 2 does not illuminate the lines 31 that are set to be inactive. Thus, the entire intensity of the light source 2 is available for illuminating the active light modulator cells 28a, 28b.

In FIG. 6, the situation in the case of a conventional exposure device according to the state of the art is shown, in comparison, to make the principle on which the present invention is based clear.

The light source 2 and the illumination optics 23 can be seen schematically. Furthermore, the light modulator 4 can be seen. The active region 32 on the light modulator 4 can be seen by means of cross-hatching. In FIG. 6, it can be seen that the active region 32 is identical with the total region of the light modulator 4. Thus, all of the lines of the light modulator 4 are active.

Furthermore, it can be seen that the entire region of the light modulator 4, which is identical with the active region 32 of the light modulator 4, is completely illuminated by means of the light source 2 and the imaging optics 23.

For the scrolling process, the complete data pattern data are transmitted for all the lines of the light modulator 4, for each exposure step and each scrolling step, respectively. Because of the large amount of data to be transmitted, it is disadvantageous that the maximal exposure speed can be limited by the data transmission rate that the light modulator 4 maximally permits, so that under some circumstances, the minimally required exposure time that results from the photochemical properties of the material 25 to be exposed is exceeded by far. This has the great disadvantage that the exposure speed clearly remains behind the photochemical limits.

With reference to FIG. 7, the functional principle of the exposure device according to the present invention will be explained. Again, the light source 2 and the illumination optics 23 can be seen schematically. It can now be seen on the light modulator 4 that a part of the total display area consists of the inactive lines 31. The active region 32 of the light modulator 4, with the active lines, now comprises merely the upper region of the light modulator 4. Furthermore, the complete region of the light modulator, in other words both the inactive lines 11 and the active region 32, is illuminated by the light source 2 and the illumination optics 23. The data pattern data to be transmitted to the light modulator 4 by way of the driver circuit 7 is clearly reduced as compared with the situation in FIG. 2, since data no longer have to be transmitted for the inactive lines 31. In this way, a significantly greater scrolling rate can be achieved, in advantageous manner, since the data pattern data that must be transmitted to the light modulator 4 per exposure step or partial scrolling step, respectively, is clearly reduced as compared with the situation shown in FIG. 6, which corresponds to the state of the art.

Finally, in FIG. 8, a state comparable to the situation in FIG. 7 is shown. Again, of the total area of the light modulator 4, the inactive lines 31 are switched to be inactive, in such a manner that no data pattern data have to be transmitted to the light modulator 4 by way of the driver circuit 27. Transmission of data pattern data by way of the driver circuit 7 only takes place for the lines of the active region 32 of the light modulator 4.

In contrast to the situation shown in FIG. 7, however, only the active region of the light modulator 4 is illuminated by the light source 2 and the illumination optics 3. The illuminated region of the light modulator 4 therefore essentially coincides with the active region 32 of the light modulator 4.

In this way, the additional advantage is achieved, as compared with the structure shown in FIG. 7, that the total radiation emitted by the light source 2 falls on the active region 32 and is therefore available for exposure of the light-sensitive material 25. In contrast, the radiation that is available for exposure of the light-sensitive material 25 in the embodiment of the invention shown in FIG. 7 is reduced by the proportion of the non-radiation of the light source 2 that falls on the inactive lines 31.

The method of operation shown in FIG. 8 is therefore preferred, since an optimum with regard to the exposure times can be achieved because of the great radiation density and the data data to be transmitted to the light modulator 4.

The use of laser diodes or a module consisting of several individual laser diodes is particularly suitable for the light source 2.

The active region 32 shown in FIGS. 6, 7, and 8 can furthermore be shifted on the total area of the light modulator 4, using the device for inactivation of lines. For example, it is possible to dispose the non-activated lines 31 both above and below the active region 32.

Because of the possibility of shifting the active region 32, uniform aging of the individual cells 8 of the light modulator 4 can be achieved. For example, in practical manner, the inactive lines 31 can be set to those lines in which there are light modulator cells 28 that contain pixels that have failed, induced by radiation. The useful lifetime of the exposure device can be extended in this manner, as compared with the conventional exposure devices.

In this way, a device for exposing light-sensitive material, particularly offset printing plates, screens for screen printing, flexo printing plates, proof materials, steel plates for punch pattern production, as well as photo paper is indicated, in which device a significantly greater exposure speed is achieved by means of a skillful reduction of the amount of data to be transmitted to the light modulator at a given data transmission rate to the light modulator 4.

Furthermore, the result can be achieved that the full light emission of the light source is available, without change, for the exposure process of the light-sensitive material, by means of the use of focusing optics in combination with a light source having a very much smaller etendue than the etendue of the total system.

### REFERENCE SYMBOL LIST

1 exposure device
2 light source
3 light coupling unit
4 light modulator
5 imaging optics
6 material to be exposed
7 laser diodes
8 individual glass fiber
9 fiber bundle
10 modulated light
11 module
12 outer housing
13 FC plug
14 cooling plate
15 cooling pipe
16 connector piece
17 control unit
18 D-sub plug
19 cooling body
20 total radiation
21 integrator rod
23 illumination optics
25 light-sensitive material
26 position transmitter
27 driver circuit
28 light modulator cell
28a light modulator cell (turned on)
28b light modulator cell (turned off)
29 imaging optics
30 device for inactivating lines
31 inactive lines
32 active region
33 field lens
34 mirror
35 lens system
36 integration lens
37 bean wave rider
38 exposure head
39 cooling water inflow
40 cooling water outflow
41 machine bed
42 axes

1-19. (canceled)

20: Exposure device (1) having a light source (2), a light modulator (4) having a plurality of lines of light-modulating cells (28), a device (27) for imaging data patterns on the light modulator (4), a device (29) for imaging the light modulator (4) on light-sensitive material (25), a device (26) for producing a relative movement between the light modulator (4) and the light-sensitive material (25), whereby the relative movement essentially runs perpendicular to the lines of light-modulating cells (28), as well as having a device (27) for scrolling a data pattern displayed on a given line of the light modulator (4) through the various lines of the light modulator (4), whereby the scrolling takes place synchronized with the relative movement, in such a manner that imaging of a data pattern displayed on the light modulator (4) on the light-sensitive material (25) is essentially stationary with reference to the light-sensitive material (25), whereby a device (30) for inactivation of a number of lines of the light modulator (4), which number can be predetermined, is provided, and the light modulator (4) can be operated by means of charging essentially only the active lines (32) with the data patterns, wherein at least one laser diode is disposed in stationary manner outside of the exposure head.

21: Device (1) according to claim 20, wherein the device (30) is configured for inactivation of a cohesive region of lines, which region can be predetermined.

22: Device (1) according to claim 21, wherein the region is configured to be shiftable.

23: Device (1) according to claim 20, wherein means (33) are provided for focusing the light source (2) essentially on the active lines of the light modulator (4).

24: Device (1) according to claim 23, wherein the means (23) for focusing are configured to be variable with reference to a focal region.

25: Device (1) according to claim 20, wherein the light source (2) has a smaller etendue than a unit formed from the light modulator (4) and the device (29) for imaging the light modulator (4) on light-sensitive material (25).

26: Device according to claim 20, wherein the light source (2) has a water cooling system.

27: Device (1) according to claim 20, wherein at least one beam waveguide (37) and one integrator rod or hollow conductor, i.e. light tunnel (21) are disposed between fiber bundle (9) and light modulator (4).

28: Device according to claim 20, wherein the laser is configured as a fiber laser or disk laser.

29: Exposure device (1) according to claim 20, wherein a light coupling unit (3) is provided and the light source (2) comprises a laser diode (7).

30: Exposure device (1) according to claim 20, wherein the laser diode (7) emits light in the range of approximately 350 nm to approximately 450 nm.

31: Exposure device (1) according to claim 20, wherein the light source (2) comprises a module (11) of several, preferably twenty laser diodes (7).

32: Exposure device (1) according to claim 20, wherein a radiation flow of each laser diode (7) is optically superimposed in the light coupling unit (3).

33: Exposure device (1) according to claim 20, wherein the light coupling unit (3) comprises individual glass fibers (8) and/or that every laser diode (7) has a separate individual glass fiber (8) assigned to it, in each instance, and/or that the individual glass fiber (8) has a diameter of approximately 125 μm and/or that the individual glass fibers (8) are brought together in the light coupling unit (3), to form a fiber bundle (9).

34: Exposure device (1) according to claim 20, wherein the outside diameter of the fiber bundle (9) amounts to approximately 650 μm.

35: Exposure device (1) according to claim 20, wherein the light source (2) comprises several modules (11).

36: Exposure device (1) according to claim 20, wherein the light coupling unit (3) comprises a light-integrating glass rod (21) and preferably has an angular, particularly a rectangular cross-section.

37: Exposure device (1) according to claim 20, wherein the radiation flow of the light source (2), preferably of each laser diode (7), is configured to be variable and/or the light source (2) is provided with means for separately turning individual laser diodes (7) on and off.
